(19) United States
(12) Patent Application Publication
(10) Pub. No.: US 2007/0195970 A1
(43) Pub. Date: Aug. 23, 2007

(54) SOUND OUTPUT DEVICE AND METHOD FOR HEARING PROTECTION

(75) Inventors: Rong-Hwa Ding, Shenzhen (CN); Ho-Leung Cheung, Shenzhen (CN); Kuan-Hong Hsieh, Shenzhen (CN)

Correspondence Address: NORTH AMERICA INTELLECTUAL PROPERTY CORPORATION, P.O. BOX 506, MERRIFIELD, VA, 22116, US

(73) Assignee: HON HAI PRECISION INDUSTRY CO., LTD., Taipei Hsien (TW)

(21) Appl. No.: 11/619,602

(22) Filed: Jan. 3, 2007

(30) Foreign Application Priority Data
Feb. 18, 2006 (CN) 200610033844.1

(51) Int. Cl. A61F 11/06; H04R 1/10

(52) U.S. Cl. 381/72; 381/74

(57) ABSTRACT

The present invention relates to a hearing protection method, includes steps of: obtaining digital audio signals from an audio signal source; sampling the digital audio signals and obtaining a plurality of amplitude values; calculating an audio energy within a predetermined time period according to the amplitude values sampled in the predetermined time period; determining whether the audio energy reaches a predetermined value; and generating a hearing protect signal when the audio energy reaches the predetermined value, thereby protecting users' hearing. The present invention also provides a corresponding sound output device for hearing protection.

### CROSS REFERENCE TO RELATED APPLICATION

This application is related to commonly-assigned copending application entitled, “SOUND OUTPUT DEVICE AND METHOD FOR HEARING PROTECTION”, filed on Dec. 5, 2006. Disclosure of the above identified application is incorporated herein by reference.

### BACKGROUND OF THE INVENTION

1. Field of the Invention

The present invention relates to a sound output device and a method for hearing protection, and particularly, to a sound output device and method for automatically reducing a current gain value or enables an alarm, when an audio energy level of digital audio signals received from an audio signal source reaches a predetermined value.

2. Description of Related Art

Developments in the digital data technology have made portable audio devices (such as MP3 players) become popular among people. When environmental noise external of the portable audio device is loud or when a favorite song is played, a user commonly increases a volume of a portable device. However users all have a physiological hearing threshold, i.e., loudness discomfort level (LDL). If the user is directly exposed to a noise volume level that is larger than the user's LDL for a long time, hearing of the user may be impaired.

In order to solve the problems mentioned, there is a common gain control system available in the market. The gain control system and method provide a gain value limit, and prevent the user from adjusting the gain value beyond the gain value limit. However, the system and method is rigid and not user friendly.

Therefore, a heretofore unaddressed need exists in the industry to overcome the aforementioned deficiencies and inadequacies.

### SUMMARY OF THE INVENTION

The present invention provides a sound output device and method for hearing protection. The sound output device and method are for receiving digital audio signals from an audio signal source, computing an audio energy level within a predetermined time period, and automatically changing a current gain value or outputting prompt sounds when the audio energy level reaches a predetermined value.

The sound output device includes an interface, a DAC and a processing unit. The interface is configured for attaching to an audio signal source. The DAC is configured for receiving digital audio signals from the audio signal source via the interface, and converting the digital audio signals to analog audio signals. The processing unit receives the digital audio signals from the audio signal source via the interface. The processing unit includes an amplitude sampling module, an energy computing module and a hearing protection module. The amplitude sampling module samples the digital audio signals at a predetermined frequency, and obtains a plurality of amplitude values of the sampled digital audio signals. The energy computing module periodically computes an audio energy within a predetermined time period according to the amplitude values sampled in the predetermined time period. The hearing protection module determines whether the audio energy reaches a predetermined value, and generates a hearing protect signal when the audio energy reaches the predetermined value.

The hearing protect method includes the steps of: receiving digital audio signals from an audio signal source; converting the analog audio signals to digital audio signals; sampling the digital audio signals to obtain a plurality of amplitude values; computing an audio energy within a predetermined time period according to the amplitude values sampled in the predetermined time period; determining whether the audio energy reaches a predetermined value; and generating a hearing protect signal when the audio energy reaches the predetermined value.

Other systems, methods, features, and advantages will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

FIG. 1 is a schematic diagram of a sound output device for hearing protection in accordance with a preferred embodiment of the present invention;

FIG. 2 is a block diagram of a hardware infrastructure of the sound output device for hearing protection of FIG. 1;

FIG. 3 is a schematic diagram of main function modules of a gain managing unit of FIG. 2;

FIG. 4 is a flowchart of a preferred method for hearing protection by utilizing the sound output device of FIG. 2;

FIG. 5 is a schematic diagram of a hardware infrastructure of a sound output device for hearing protection in accordance with an alternative embodiment of the present invention; and

FIG. 6 is a schematic diagram of main function modules of a gain managing unit of FIG. 5.

### DETAILED DESCRIPTION OF THE INVENTION

In the following embodiments, for simplicity, a hearing protection function incorporated in a sound output device, such as an earphone, is depicted. The following detailed description of the embodiments is made with reference to the attached drawings.

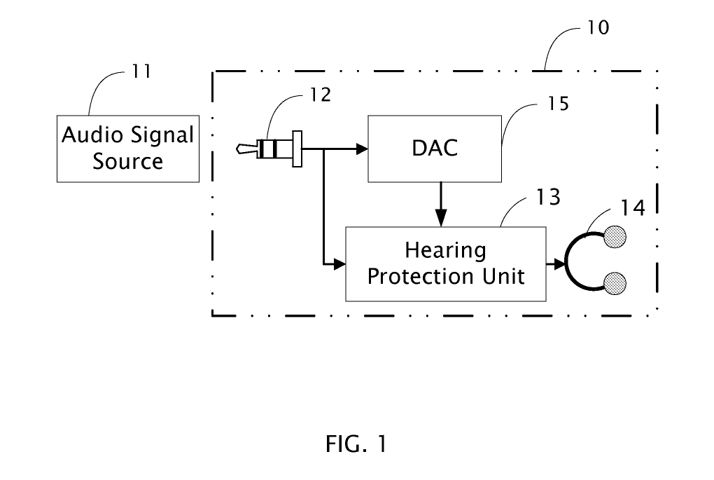

FIG. 1 is a schematic diagram of a hardware infrastructure of a sound output device for hearing protection in accordance with a preferred embodiment of the present invention. The sound output device 10 includes an interface 12, a hearing protection unit 13, a digital-to-analog converter (DAC) 15, and a sound transducer 14. The sound output device 10 receives digital audio signals from an audio signal source 11 via the interface 12, and sends the digital audio signals to the hearing protection unit 13 and the DAC 15 respectively. The audio signal source 11 can be a music player, a radio player, a TV set, and so on.

The DAC 15 converts the digital audio signals to analog audio signals, and sends the analog audio signals to the hearing protection unit 13.

The hearing protection unit 13 calculates an audio energy of the digital audio signals within a predetermined time period. If the audio energy reaches a predetermined value, the hearing protection unit 13 may automatically change a current gain value to a reduced gain value, or outputs prompt signals to the sound transducer 14 to reproduce prompt sounds corresponding to the prompt signals. The prompt sounds alerts a user to manually change the current gain value to the reduced gain value, thus, preventing hearing impairment of the user.

The sound transducer 14 further reproduces sounds corresponding to the analog audio signals converted by the DAC 15.

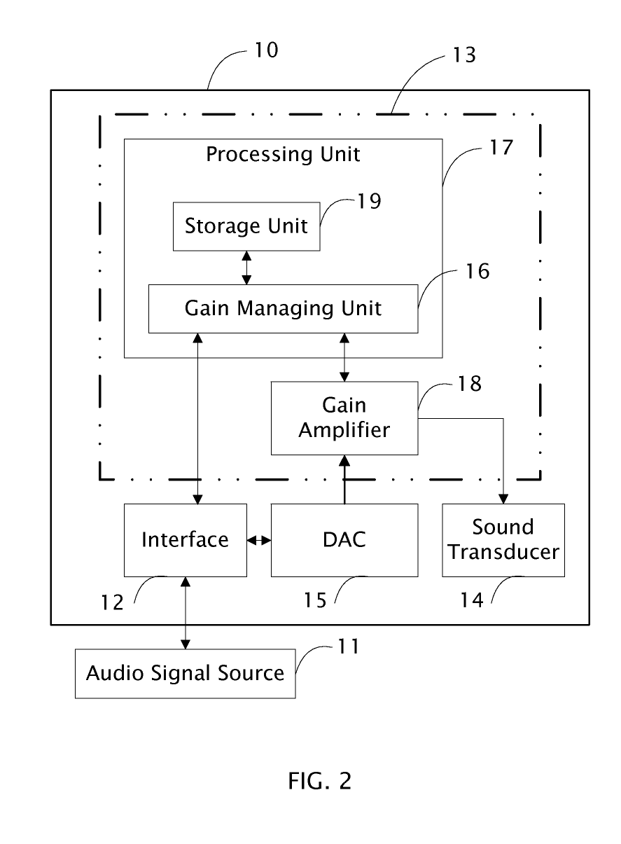

FIG. 2 is a block diagram of a hardware infrastructure of the sound output device for hearing protection of FIG. 1. The hearing protection unit 13 includes a processing unit 17 and a gain amplifier 18. The processing unit 17 includes a gain managing unit 16 and a storage unit 19. The storage unit 19 stores a default gain value. The storage unit 19 may be a flash storage, a hard disk driver, and the like. The processing unit 17 controls elements of the hearing protection unit 13, i.e., the gain managing unit 16, the gain amplifier 18 and the storage unit 19.

The DAC 15 receives the digital audio signals from the audio signal source 11 via the interface 12, converts the digital audio signals to analog audio signals, and sends the analog audio signals to the gain amplifier 18.

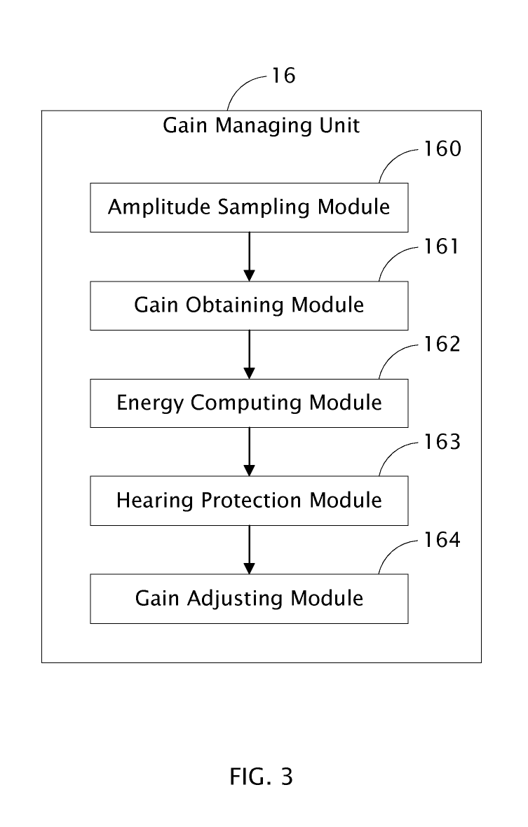

Referring to FIG. 3, the gain managing unit 16 includes an amplitude sampling module 160, a gain obtaining module 161, an energy computing module 162, a hearing protection module 163, and a gain adjusting module 164. The amplitude sampling module 160 samples the digital audio signals received from the audio signal source 11 at a predetermined frequency continuously, and obtains a plurality of amplitude values of the sampled digital audio signals.

The gain obtaining module 161 reads the default gain value from the storage unit 19. The energy computing module 162 periodically calculates an audio energy within a predetermined time period by: Q=[Σ(mi*V)²/N]^{1/2}*T, where V represents the default gain value, T represents the predetermined time period, Q represents the audio energy of the digital audio signals within the predetermined time period, N represents a count of the amplitude values sampled in the predetermined time period, i is any natural number from 1 to N, and mi represents the amplitude values sampled in the predetermined time period.

The hearing protection module 163 determines whether the audio energy reaches the predetermined value, and if the audio energy reaches the predetermined value, generates a hearing protect signal, and sends the hearing protect signal to the gain adjusting module 164.

The gain adjusting module 164 is configured for receiving the hearing protect signal from the hearing protection module 163. After receiving the hearing protect signal, the gain adjusting module 164 automatically changes the default gain value to reduced gain value, and signals the gain amplifier 18 to amplify the analog audio signals received from the DAC 15 according to the reduced gain value, thereby yielding amplified analog audio signals. The gain adjusting module 164 updates the default gain value stored in the storage unit 19 with the reduced gain value, and sends the amplified analog audio signals to the sound transducer 14.

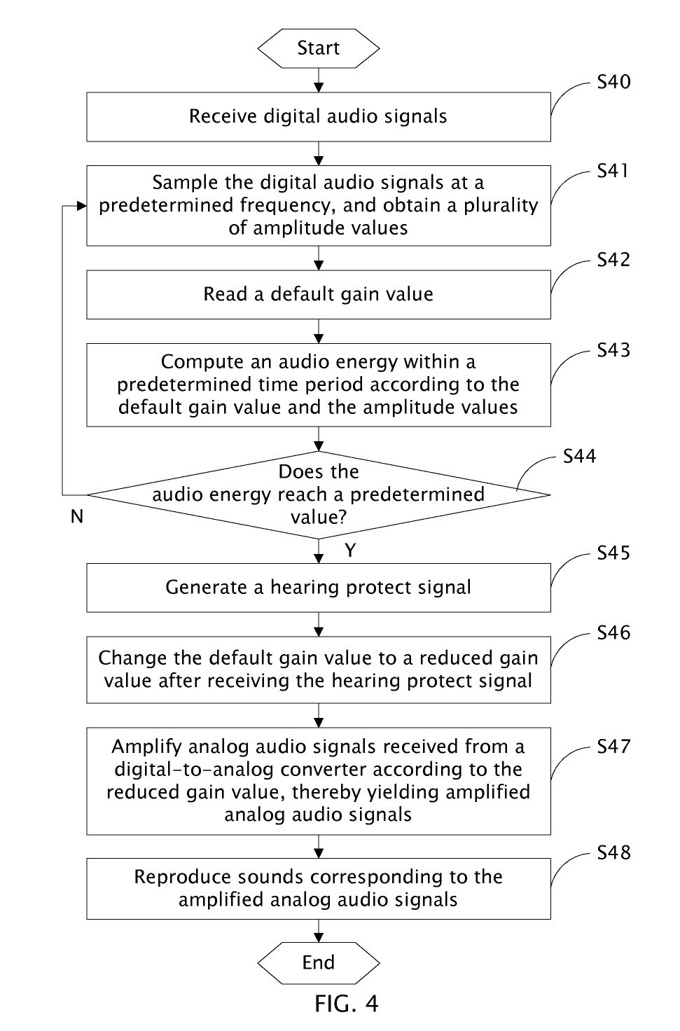

FIG. 4 is a flowchart of a preferred method for hearing protection by utilizing the sound output device of FIG. 2. In step S40, the gain managing unit 16 receives the digital audio signals from the audio signal source 11 via the interface 12.

In step S41, the amplitude sampling module 160 samples the digital audio signals, and obtains a plurality of amplitude values of the sampled digital audio signals.

In step S42, the gain obtaining module 161 reads the default gain value from the storage unit 19.

In step S43, the energy computing module 161 computes the audio energy within the predetermined time period according to the default gain value and the plurality of amplitude values sampled in the predetermined time period.

In step S44, the hearing protection module 163 determines whether the audio energy reaches the predetermined value.

If the audio energy does not reach the predetermined value, the procedure turns to step S41; if the audio energy reaches the predetermined value, in step S45, the hearing protection module 163 generates the hearing protect signal, and sends the hearing protect signal to the gain adjusting module 164.

In step S46, after receiving the hearing protect signal, the gain adjusting module 164 automatically changes the default gain value to the reduced gain value, and updates the default gain value stored in the storage unit 19 with the reduced gain value.

In step S47, the gain adjusting module 164 signals the gain amplifier 18 to amplify the analog audio signals received from the DAC 15 according to the reduced gain value, thereby yielding amplified analog audio signals; the gain amplifier 18 sends the amplified analog audio signals to the sound transducer 14.

In step S48, the sound transducer 14 receives the amplified analog audio signals, and reproduces sounds corresponding to the amplified analog audio signals.

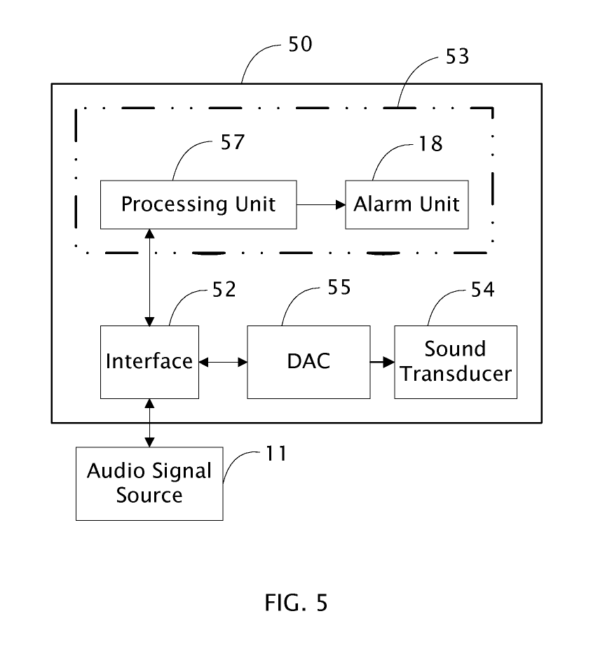

FIG. 5 is a block diagram of a hardware infrastructure of the sound output device for hearing protection in accordance with an alternative embodiment of the present invention. The sound output device 50 includes an interface 52, a hearing protection unit 53, a DAC 55, and a sound transducer 54. The hearing protection unit 53 includes a processing unit 57 and an alarm unit 58. The alarm unit 58 may be an acoustical alarm unit such as a buzzer, or a visual alarm unit such as an LED (light-emitting diode).

The DAC 55 receives the digital audio signals from the audio signal source 11 via the interface 52, converts the digital audio signals to analog audio signals, and sends the analog audio signals to the sound transducer 54. The sound transducer 54 reproduces sounds corresponding to the analog audio signals.

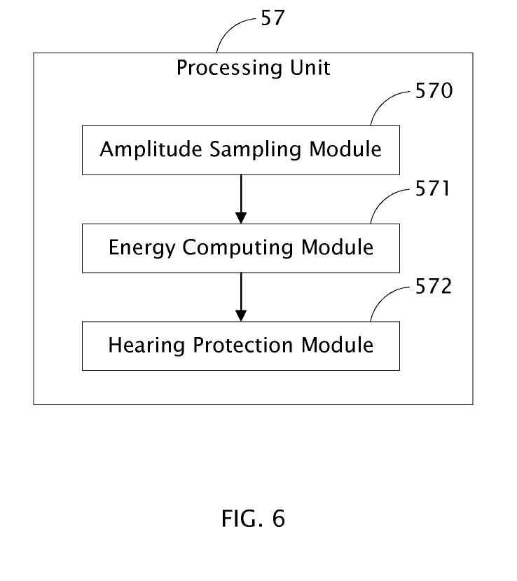

The processing unit 57 receives the digital audio signals from the audio signal source 11 via the interface 52. Referring to FIG. 6, the processing unit 57 includes an amplitude sampling module 570, an energy computing module 571 and a hearing protection module 572.

The amplitude sampling module 570 samples the digital audio signals at a predetermined frequency, and obtains a plurality of amplitude values of the sampled digital audio signals.

The energy computing module 571 periodically calculates an audio energy within the predetermined time period by: Q=[Σ(mi)²/N]^{1/2}*T, wherein T represents the predetermined time period, Q represents the audio energy of the digital audio signals within the predetermined time period, N represents a count of the amplitude values sampled within the predetermined time period, i is any natural number from 1 to N, and mi represents the amplitude values sampled within the predetermined time period.

The hearing protection module 572 determines whether the audio energy reaches the predetermined value, and if the audio energy reaches the predetermined value, generates a hearing protect signal, and sends the hearing protect signal to the alarm unit 58.

After receiving the hearing protect signal, the alarm unit 58 outputs prompt sounds for alerting the user to change the current gain value. The prompt sounds may be acoustical reminding signals or visual alarm signals.

It is understood that the invention may be embodied in other forms without departing from the spirit thereof. Thus, the present examples and embodiments are to be considered in all respects as illustrative and not restrictive, and the invention is not to be limited to the details given herein.

What is claimed is:

1. A sound output device for hearing protection comprising:
an interface configured for attaching to an audio signal source;
a DAC configured for receiving digital audio signals from the audio signal source via the interface, and converting the digital audio signals to analog audio signals; and
a processing unit configured for receiving the digital audio signals from the audio signal source via the interface, comprising:
an amplitude sampling module for sampling the digital audio signals at a predetermined frequency, and obtaining a plurality of amplitude values of the digital audio signals;
an energy computing module for periodically calculating an audio energy of a predetermined time period according to the amplitude values sampled within the predetermined time period; and
a hearing protection module for determining whether the audio energy reaches a predetermined value, and generating a hearing protect signal when the audio energy reaches the predetermined value.

2. The sound output device according to claim 1, further comprising a storage unit configured for storing a default gain value, and a gain amplifier configured for amplifying the analog audio signals received from the DAC.

3. The sound output device according to claim 2, wherein the processing unit further comprises a gain obtaining module, the gain obtaining module for reading the default gain value acting as a parameter for calculating the audio energy.

4. The sound output device according to claim 3, wherein the audio energy is calculated by: Q=[Σ(mi*V)²/N]^{1/2}*T, where V represents the default gain value, T represents the predetermined time period, Q represents the audio energy of the predetermined time period, N represents a count of the amplitude values sampled in the predetermined time period, i is any natural number from 1 to N, and mi represents the amplitude values sampled within the predetermined time period.

5. The sound output device according to claim 2, wherein the processing unit further comprises a gain adjusting module, the gain adjusting module for receiving the hearing protect signal, automatically changing the default gain value to a reduced gain value, signaling the gain amplifier to amplify the analog audio signals received from the DAC according to the reduced gain value, and updating the default gain value stored in the storage unit with the reduced gain value.

6. The sound output device according to claim 1, further comprising an alarm unit configured for receiving the hearing protect signal, and generating prompt sounds to alert a user to manually reduce the default gain value.

7. The sound output device according to claim 6, wherein the audio energy is calculated by: Q=[Σ(mi)²/N]^{1/2}*T, where T represents the predetermined time period, Q represents the audio energy of the predetermined time period, N represents a count of the amplitude values sampled within the predetermined time period, i is any natural number from 1 to N, and mi represents the amplitude values sampled within the predetermined time period.

8. The sound output device according to claim 6, wherein the prompt sounds are selected from the group consisting of visual reminding sounds and acoustical reminding sounds.

9. A hearing protection method of a sound output device, the method comprising the steps of:
receiving digital audio signals from an audio signal source;
sampling the digital audio signals to obtain a plurality of amplitude values;
computing an audio energy of a predetermined time period according to the amplitude values sampled within the predetermined time period;
determining whether the audio energy reaches a predetermined value; and
generating a hearing protect signal when the audio energy reaches the predetermined value.

10. The method according to claim 9, further comprising the step of: reading a default gain value acting as a parameter for calculating the audio energy.

11. The method according to claim 10, wherein the audio energy is calculated by: Q=[Σ(mi*V)²/N]^{1/2}*T, where V represents the default gain value, T represents the predetermined time period, Q represents the audio energy of the predetermined time period, N represents a count of the amplitude values sampled within the predetermined time period, i is any natural number from 1 to N, and mi represents the amplitude values sampled within the predetermined time period.

12. The method according to claim 9, further comprising the steps of: after receiving the hearing protect signal, automatically changing the default gain value to a reduced gain value, converting the received digital audio signals to analog audio signals, amplifying the analog audio signals according to the reduced gain value, and updating the default gain value with the reduced gain value.

13. The method according to claim 9, further comprising the steps of: receiving the hearing protect signal, and generating prompt sounds to alert a user.

14. The method according to claim 13, wherein the audio energy is calculated by: Q=[(mi)²/N]^{1/2}*T, where T represents the predetermined time period, Q represents the audio energy of the predetermined time period T, N represents a count of the amplitude values sampled within the predetermined time period, i is any natural number from 1 to N, and mi represents the amplitude values sampled within the predetermined time period.

15. The method according to claim 13, wherein the prompt sounds are selected from the group consisting of visual reminding sounds and acoustical reminding sounds.
